(19) United States
(12) Patent Application Publication
(10) Pub. No.: US 2020/0357520 A1
(43) Pub. Date: Nov. 12, 2020

(54) DIAGNOSIS SUPPORT APPARATUS

(71) Applicant: CANON MEDICAL SYSTEMS CORPORATION, Otawara-shi (JP)

(72) Inventors: Katsuhiko FUJIMOTO, Saitama (JP); Satoshi IKEDA, Edinburgh (GB); Keisuke HASHIMOTO, Nasushiobara (JP); Mariko SHIBATA, Nasushiobara (JP); Narumi SASAYAMA, Nasushiobara (JP)

(73) Assignee: CANON MEDICAL SYSTEMS CORPORATION, Otawara-shi (JP)

(21) Appl. No.: 16/869,713

(22) Filed: May 8, 2020

(30) Foreign Application Priority Data
May 10, 2019 (JP) 2019-089492

(51) Int. Cl. G16H 50/20; A61B 5/00; G16B 20/00; G16H 10/40; G16H 50/30; G16H 50/70; G16H 50/50; G16H 15/00

(52) U.S. Cl. CPC G16H 50/20; A61B 5/4842; G16B 20/00; G16H 15/00; G16H 50/30; G16H 50/70; G16H 50/50; G16H 10/40

(57) ABSTRACT

A diagnosis support apparatus according to an embodiment includes a memory and processing circuitry. The memory stores therein a plurality of types of living body information including gene expression and mutation information, epigenetic environment influence information, protein expression information, signal transmission information, immune function information, endocrine function information, pathological information, image diagnosis information, physiological information, and body findings and symptom information of a subject. The processing circuitry determines a living body state of the subject on the basis of a plurality of analysis results obtained by analysis of the types of living body information.

### CROSS-REFERENCE TO RELATED APPLICATIONS

This application is based upon and claims the benefit of priority from Japanese Patent Application No. 2019-089492, filed on May 10, 2019; the entire contents of which are incorporated herein by reference.

### FIELD

Embodiments described herein relate generally to a diagnosis support apparatus.

### BACKGROUND

In prior art, many diagnostic and inspection techniques exist, such as gene diagnosis using a next generation sequencer (NGS) or a genetic panel, in-vitro diagnostics (IVD) inspection, protein analysis, antibody check, histopathological diagnosis, radiation image diagnosis, image diagnosis other than radiation image diagnosis, and non-image inspection.

In recent years, in the field of cancer, companion diagnosis or the like has been performed increasingly. Companion diagnosis is performed to properly select remedies, such as new anticancer agents, molecular target drugs, and immune checkpoint inhibitors, by inspection using NGS or a genetic panel.

In a cancer genetic panel inspection, it is advanced in Japan to make a mechanism for an attempt of profiling using AI (Cognitive Computing System) or the like, such as IBM Watson based on tens of millions of papers. In addition, an attempt of comprehensive genomic profiling using tumor mutational burden (TMB) and/or microsatellite instability (MSI) using Roche-foundation medicines is advanced.

In conventional diagnosis and inspection, for example, diagnosis or clinical determination based on individual diagnosis or an individual inspection result is performed, such as diagnosis using gene profiling. As another example, determination is made in a complex manner on the basis of a plurality of diagnostic results in accordance with guidelines and the doctor's experience.

However, with conducting and presenting these limited diagnosis and inspections individually, it is difficult for the doctor to determine the state (living body state) of the living body of the subject with high accuracy.

### BRIEF DESCRIPTION OF THE DRAWINGS

FIG. 1 a diagram illustrating a configuration example of a diagnosis support apparatus according to a first embodiment;

FIG. 2 is a diagram illustrating an example of a data structure of a living body state determination database;

FIG. 3 is a diagram for explaining an example of a method for calculating a relation degree from quantitative scores in the first embodiment;

FIG. 4 is a diagram illustrating an example of display of the relation degrees and total reliabilities according to the first embodiment;

FIG. 5 is a flowchart illustrating a process of an example of processing performed with the diagnosis support apparatus according to the first embodiment;

FIG. 6 is a diagram illustrating an example of warning information according to a third modification of the first embodiment; and

FIG. 7 is a diagram for explaining an example of a method for estimating a living body state according to a second embodiment.

### DETAILED DESCRIPTION

A diagnosis support apparatus according to an embodiment includes a memory and processing circuitry. The memory stores therein a plurality of types of living body information including gene expression and mutation information, epigenetic environment influence information, protein expression information, signal transmission information, immune function information, endocrine function information, pathological information, image diagnosis information, physiological information, and body findings and symptom information of a subject. The processing circuitry determines a living body state of the subject on the basis of a plurality of analysis results obtained by analysis of the types of living body information.

The following is a detailed explanation of embodiments of a diagnosis support apparatus with reference to drawings. The diagnosis support apparatus according to the present application is not limited with the embodiments described hereinafter.

### First Embodiment

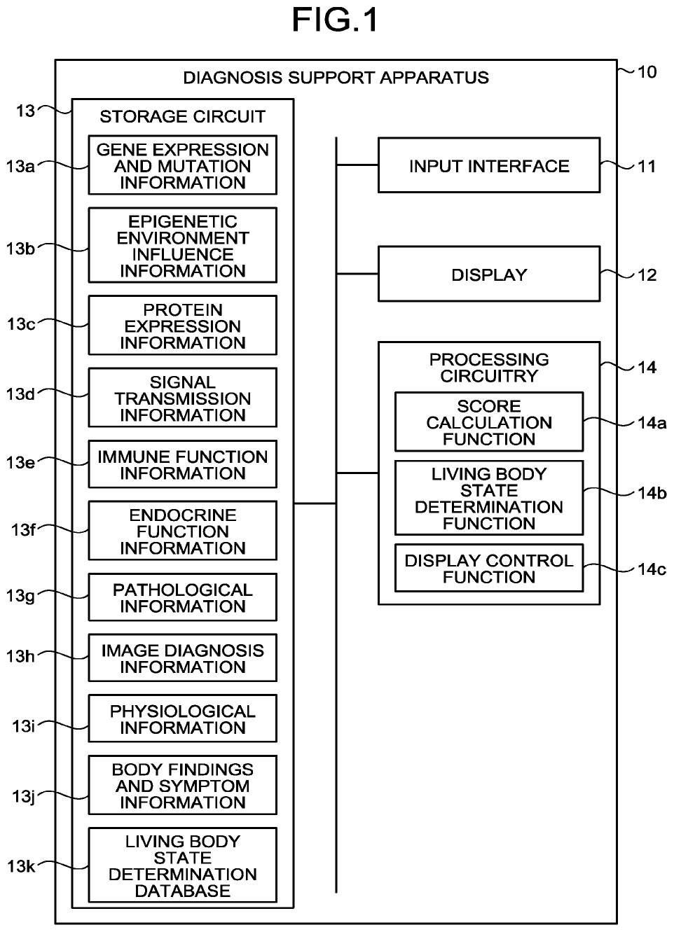

FIG. 1 is a diagram illustrating a configuration example of a diagnosis support apparatus 10 according to a first embodiment. The diagnosis support apparatus 10 is an apparatus supporting diagnosis of the disease (illness) of the subject by an operator (such as the doctor). For example, the diagnosis support apparatus 10 is achieved with a computer equipment, such as a server, a work station, a personal computer, and a tablet terminal.

As illustrated in FIG. 1, the diagnosis support apparatus 10 includes an input interface 11, a display 12, a storage circuit 13, and processing circuitry 14.

The input interface 11 is connected with the processing circuitry 14, and receives various instructions and information input operations from the operator. Specifically, the input interface 11 converts an input operation received from the operator into an electrical signal, and outputs the electrical signal to the processing circuitry 14. For example, the input interface 11 is achieved with at least one of a trackball, a switch button, a mouse, a keyboard, a touch pad to perform an input operation by touch on the operating surface, a touch screen in which a display screen is united with a touch pad, a noncontact input circuit using an optical sensor, and a sound input circuit. In the present specification, the input interface 11 is not limited to an element including a physical operating component, such as a mouse and a keyboard. For example, examples of the input interface 11 include electrical signal processing circuitry receiving an electrical signal corresponding to an input operation from an external input device provided as an element separated from the apparatus, and outputting the electrical signal to the processing circuitry 14.

The display 12 is connected with the processing circuitry 14, and displays various types of information and images. Specifically, the display 12 converts information and image data transmitted from the processing circuitry 14 into display electrical signals, and outputs the display electrical signals. For example, the display 12 is achieved with a liquid crystal monitor, a cathode ray tube (CRT) monitor, a touch panel, or the like. The display 12 serves as an example of the display.

The storage circuit 13 is connected with the processing circuitry 14, and stores therein various types of data and/or information. For example, the storage circuit 13 is achieved with a semiconductor memory element, such as a flash memory, a hard disk, or an optical disk. Specifically, the storage circuit 13 is achieved with a memory or the like. The storage circuit 13 serves as an example of the storage unit.

The storage circuit 13 stores therein gene expression and mutation information 13a, epigenetic environment influence information 13b, protein expression information 13c, signal transmission information 13d, immune function information 13e, endocrine function information 13f, pathological information 13g, image diagnosis information 13h, physiological information 13i, body findings and symptom information 13j, and a living body state determination database 13k.

The gene expression and mutation information 13a is, for example, living body information indicating expression quantity and mutation quantity of a specific gene of the subject serving as the diagnosis target. For example, the gene expression and mutation information 13a is obtained by gene inspection or the like using a sequencer, a gene panel, or the like. The obtained gene expression and mutation information 13a is stored in the storage circuit 13.

The epigenetic environment influence information 13b is, for example, living body information indicating control of gene expression caused by an acquired environmental factor providing an influence on epigenome of the subject, such as histone modification and methylation. For example, the operator inquires of the subject about the number of cigarettes that the subject smokes per day, or the quantity of ultraviolet rays to which the subject is exposed per day. As another example, the phenomenon resulting therefrom, such as histone modification and methylation, is detected, and the obtained environmental factor information and/or information indicating the inspection result is stored in the storage circuit 13 as the epigenetic environment influence information 13b.

The protein expression information 13c is living body information relating to protein expression of the subject. For example, the protein expression information 13c is information indicating a ratio of quantity of specific protein in the blood of the subject or the like to quantity serving as a standard of the specific protein. The quantity of the specific protein of the subject is obtained by in-vitro biomarker inspection or spectroscopic analysis and with a mass spectrometer (such as mass spectroscopy). The quantity serving as the standard of the specific protein is, for example, quantity of the specific protein when the subject is in a healthy state. The obtained information indicating the ratio of the quantity of the specific protein to the quantity serving as the standard of the specific protein is stored in the storage circuit 13 as the protein expression information 13c.

The signal transmission information 13d is living body information relating to signal transmission between cells and in the cell of the subject. The signal transmission information 13d is obtained with an antibody array or the like. The obtained signal transmission information 13d is stored in the storage circuit 13.

The immune function information 13e is living body information relating to the immune function of the subject. For example, the immune function information 13e is information indicating the number of leukocytes per unit quantity of the blood of the subject. The number of leukocyte like this is obtained by blood test or the like. The immune function information 13e may be information indicating the expression quantity and the mutation quantity of the specific gene relating to immunity, or information indicating the expressed antibody quantity itself. Such information is obtained with a sample inspection device, antibody test, a gene chip, or the like. The obtained immune function information 13e is stored in the storage circuit 13.

The endocrine function information 13f is living body information relating to the endocrine function of the subject. For example, the endocrine function information 13f is information indicating the quantity of specific hormone per unit quantity of the blood of the subject. Such information indicating the quantity of the specific hormone is obtained by blood test or the like.

The pathological information 13g is various types of living body information of the subject obtained by pathological test or cytodiagnosis test. For example, in a pathological test, tissue or cells of a tumor are collected, and it is determined whether the collected tissue or cells are malignant. The information indicating the result of determination like this is stored in the storage circuit 13 as the pathological information 13g.

The image diagnosis information 13h is living body information obtained with a radiation diagnostic image in which the organ of the subject is drawn and by image analysis of the radiation diagnostic image. For example, in the inspection, the doctor performs image diagnosis on a CT image in which the heart of the subject is drawn. In such image diagnosis, the doctor diagnoses whether the heart has any abnormality. For example, the doctor pays attention to the aortic valve, and diagnoses whether the subject suffers from the heart valve disease, and/or diagnoses whether the subject suffers from the aortic stenosis. The information indicating the diagnosis result is stored in the storage circuit 13 as the image diagnosis information 13h. The image diagnosis may include image diagnosis of images other than CT images. For example, the image diagnosis may include image diagnosis of at least one image of an MR image obtained with a magnetic resonance imaging (MRI) apparatus, an X-ray diagnostic image obtained with an X-ray diagnostic apparatus, an ultrasonic diagnostic image obtained with an ultrasonic diagnostic apparatus, and a nuclear medicine diagnostic image obtained with a nuclear medicine diagnostic apparatus.

The physiological information 13i is, for example, living body information relating to the heart based on the electrocardiographic waveform of the subject obtained with an electrocardiogram. In electrocardiography, the electrocardiographic waveform of the subject is obtained with an electrocardiograph. In addition, information relating to the heart, such as R-R interval, is obtained from the electrocardiographic waveform. The information relating to the heart like this is stored in the storage circuit 13 as the physiological information 13i. In addition, the physiological information 13i also includes electroencephalogram information, information of the respiration monitor, and information indicating various physiological phenomena, such as the body temperature and the blood pressure.

The body findings and symptom information 13j is living body information indicating the body findings and the symptom obtained by inquiry for the subject. For example, the doctor obtains the body findings and symptom by performing inquiry on the subject. For example, the body findings include information of the human body level, such as the height and the weight of the subject. The information indicating the body findings and the symptom obtained like this is stored in the storage circuit 13 as the body findings and symptom information 13j.

As described above, the storage circuit 13 stores therein the gene expression and mutation information 13a, the epigenetic environment influence information 13b, the protein expression information 13c, the signal transmission information 13d, the immune function information 13e, the endocrine function information 13f, the pathological information 13g, the image diagnosis information 13h, the physiological information 13i, and the body findings and symptom information 13j. As described above, the storage circuit 13 stores therein living body information of the gene level, living body information of the molecular and cell level, living body information of the organ level, and living body information of the human body level of the subject. Specifically, the storage circuit 13 stores therein a plurality of types of comprehensive living body information ranging from the gene level to the human body level of the subject. When it is explained with a specific example, the storage circuit 13 stores therein a plurality of types of living body information including living body information relating to the gene of the subject, living body information relating to the protein, living body information relating to signal transmission, living body information relating to the endocrine function, living body information relating to the immune function, living body information relating to the influence of the environment, and living body information relating to the human body.

The living body state determination database 13k is a database used when the living body state is determined.

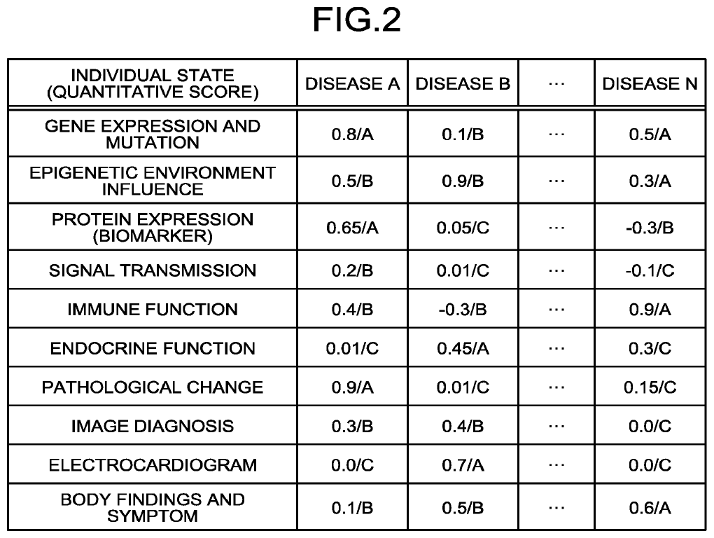

FIG. 2 is a diagram illustrating an example of a data structure of the living body state determination database 13k. In FIG. 2, a quantitative score described later and calculated from the gene expression and mutation information 13a is registered in the item “gene expression and mutation”. A quantitative score described later and calculated from the epigenetic environment influence information 13b is registered in the item “epigenetic environment influence”. A quantitative score described later and calculated from the protein expression information 13c is registered in the item “protein expression (biomarker)”. A quantitative score described later and calculated from the signal transmission information 13d is registered in the item “signal transmission”.

A quantitative score described later and calculated from the immune function information 13e is registered in the item “immune function”. A quantitative score described later and calculated from the endocrine function information 13f is registered in the item “endocrine function”. A quantitative score described later and calculated from the pathological information 13g is registered in the item “pathological change”. A quantitative score described later and calculated from the image diagnosis information 13h is registered in the item “image diagnosis”. A quantitative score described later and calculated from the physiological information 13i is registered in the item “electrocardiogram”. A quantitative score described later and calculated from the body findings and symptom information 13j is registered in the item “body findings and symptom”.

The living body state determination database 13k registers therein correlation coefficients each indicating correlation (mutual relation) between each of disease A to disease N and each of a plurality of types of living body information. The living body state determination database 13k also registers therein reliability indicating likelihood of each of the correlation coefficients. For example, in the example of FIG. 2, supposing that the correlation coefficient is “R” and the reliability is “S”, the correlation coefficient and the reliability are registered with the expression “R/S”, for each combination of the disease and the living body information, in the living body state determination database 13k.

For example, the living body state determination database 13k indicates that the correlation coefficient indicating correlation between the gene expression and mutation information 13a and “disease A” is “0.8”, in the combination of the gene expression and mutation information 13a serving as the source of the quantitative score registered in the item “gene expression and mutation” and “disease A”. The same is applicable in other combinations of the disease and the living body information.

In the present embodiment, for example, the range of the correlation coefficient is a range from “−1” to “1”. For example, the following is an explanation of the case where the correlation coefficient between correlation between a disease and a certain type of living body information has a positive value. In this case, as the correlation coefficient becomes close to “1”, the possibility of contraction of the disease increases, and the symptoms of the disease become graver, with the living body information of the type serving as the factor.

In addition, the following is an explanation of the case where the correlation coefficient between correlation between a disease and a certain type of living body information has a negative value. In this case, as the correlation coefficient becomes close to “−1”, the possibility of unlikeliness in contraction of the disease increases, with the living body information of the type serving as the factor.

In addition, when the correlation coefficient indicating correlation between a disease and a certain type of living body information is “0” or a value close to “0”, the correlation coefficient indicates that the living body information of the type has no correlation with the disease.

In addition, for example, the living body state determination database 13k indicates that the reliability indicating likelihood of the correlation coefficient “0.8” indicating correlation between the gene expression and mutation information 13a and “disease A” is “A”. The same is applicable to the other reliabilities. In the present embodiment, the reliability is expressed with one of “A”, “B”, and “C”. For example, the reliability “A” is reliability higher than the reliability “B”, and the reliability “B” is reliability higher than the reliability “C”. The reliabilities “A”, “B”, and “C” indicate numerical values. For example, the reliability “A” is “1.0”, the reliability “B” is “0.7”, and the reliability “C” is “0.3”. For example, the reliability is also determined based on the degree of scientific grounds existing for the correlation coefficient. As another example, the reliability may be determined based on the guideline, the details of the clinical researches registered in the database, the contents of papers, and the like.

The processing circuitry 14 controls operations of the diagnosis support apparatus 10, in accordance with an input operation received from the operator via the input interface 11. For example, the processing circuitry 14 is achieved with a processor.

The processing circuitry 14 includes a score calculation function 14a, a living body state determination function 14b, and a display control function 14c. The score calculation function 14a serves as an example of the analysis unit. The living body state determination function 14b serves as an example of the determination unit. The display control function 14c serves as an example of the display controller. The score calculation function 14a, the living body state determination function 14b, and the display control function 14c will be described later.

For example, processing functions of the score calculation function 14a, the living body state determination function 14b, and the display control function 14c serving as constituent elements of the processing circuitry 14 illustrated in FIG. 1 are stored in the storage circuit 13, in the form of computer programs executable with a computer. The processing circuitry 14 reads each of the computer programs from the storage circuit 13, and executes the read computer program to achieve the function corresponding to the computer program. In other words, the processing circuitry 14 in the state of reading each of the computer programs has each of the functions illustrated in the processing circuitry 14 of FIG. 1.

All the processing functions of the score calculation function 14a, the living body state determination function 14b, and the display control function 14c may be stored in the storage circuit 13, in the form of a single computer program executable with a computer. For example, such a computer program is also referred to as diagnosis support program. In this case, the processing circuitry 14 reads the diagnosis support program from the storage circuit 13, and executes the read diagnosis support program to achieve the score calculation function 14a, the living body state determination function 14b, and the display control function 14c corresponding to the diagnosis support program.

The term “processor” used in the explanation described above means a circuit, such as a central processing unit (CPU), a graphics processing unit (GPU), application specific integrated circuit (ASIC), and a programmable logic device (such as a simple programmable logic device (SPLD), a complex programmable logic device (CPLD), and a field programmable gate array (FPGA)). The processor achieves the function by reading and executing a computer program stored in the storage circuit 13. Instead of storing a computer program in the storage circuit 13, a structure may be adopted to directly integrate a computer program in the circuit of the processor. In this case, the processor achieves the function by reading and executing a computer program integrated in the circuit. The processor according to the present embodiment is not limited to the case of being configured as a single circuit, but a plurality of independent circuits may be combined to be configured as a single processor and achieve the function.

An example of the configuration of the diagnosis support apparatus 10 according to the first embodiment has been described above. As a result of diligent researches, the inventors of the present application have found that all diseases are related to complicated factors including hereditary factors and non-hereditary factors. In addition, the inventors of the present application have found that determination of the living body state of the subject using a plurality of types of comprehensive living body information ranging from the gene level to the human body level of the subject enables acquisition of the determination result with high accuracy. For this reason, the diagnosis support apparatus 10 according to the first embodiment executes various types of processing explained hereinafter using a plurality of types of comprehensive living body information ranging from the gene level to the human body level of the subject, to determine the living body state with high accuracy.

The score calculation function 14a performs analysis to determine the degree of abnormality of the living body information for each of a plurality of types of living body information stored in the storage circuit 13. When it is explained with a specific example, the score calculation function 14a performs analysis to obtain a score (quantitative score) indicating the degree of abnormality of the living body information for each of the types of living body information, and obtains a quantitative score as an analysis result. For example, the score calculation function 14a calculates a quantitative score falling within the range of “0” to “1”. For example, the quantitative score indicates that the degree of the abnormality state indicated with the living body information increases as the value becomes close to “1”. The score calculation function 14a calculates such a quantitative score for each of the gene expression and mutation information 13a, the epigenetic environment influence information 13b, the protein expression information 13c, the signal transmission information 13d, the immune function information 13e, the endocrine function information 13f, the pathological information 13g, the image diagnosis information 13h, the physiological information 13i, and the body findings and symptom information 13j. The quantitative score is used when the living body state is determined with the living body state determination function 14b described later.

When it is explained with an example, the score calculation function 14a calculates, as an analysis result, a quantitative score having a value that increases as the expression quantity and the mutation quantity of the specific gene indicated with the gene expression and mutation information 13a increase, as analysis for the gene expression and mutation information 13a. The score calculation function 14a registers the calculated quantitative score in the item “gene expression and mutation” in the living body state determination database 13k.

In addition, the score calculation function 14a calculates, as an analysis result, a quantitative score having a value that increases as the number of cigarettes or the quantity of ultraviolet rays indicated with the epigenetic environment influence information 13b increases or the score of methylation or the like increases, as analysis for the epigenetic environment influence information 13b. The score calculation function 14a registers the calculated quantitative score in the item “epigenetic environment influence” in the living body state determination database 13k.

In addition, the score calculation function 14a calculates, as an analysis result, a quantitative score having a value that increases as the ratio indicated with the protein expression information 13c becomes distant from “1”, as analysis for the protein expression information 13c. The score calculation function 14a registers the calculated quantitative score in the item “protein expression (biomarker)” in the living body state determination database 13k.

In addition, the score calculation function 14a calculates, as an analysis result, a quantitative score having a value that increases as the degree of abnormality indicated with information relating to signal transmission indicated with the signal transmission information 13d increases, as analysis for the signal transmission information 13d. The score calculation function 14a registers the calculated quantitative score in the item “signal transmission” in the living body state determination database 13k.

In addition, the score calculation function 14a calculates, as an analysis result, a quantitative score having a value that increases as the degree of abnormality indicated with the number of leukocytes indicated with the immune function information 13e increases, as analysis for the immune function information 13e. When the immune function information 13e indicates the expression quantity and the mutation quantity of the specific gene relating to immunity, the score calculation function 14a calculates, as an analysis result, a quantitative score having a value that increases as the expression quantity and the mutation quantity of the specific gene relating to immunity increase. The score calculation function 14a registers the calculated quantitative score in the item “immune function” in the living body state determination database 13k.

In addition, the score calculation function 14a calculates, as an analysis result, a quantitative score having a value that increases as the degree of abnormality indicated with the quantity of specific hormone indicated with the endocrine function information 13f increases, as analysis for the endocrine function information 13f. The score calculation function 14a registers the calculated quantitative score in the item “endocrine function” in the living body state determination database 13k.

In addition, the score calculation function 14a calculates, as an analysis result, a quantitative score having a value that increases as the degree of abnormality indicated with the determination result indicated with the pathological information 13g increases, as analysis for the pathological information 13g. The score calculation function 14a registers the calculated quantitative score in the item “pathological change” in the living body state determination database 13k.

In addition, the score calculation function 14a calculates, as an analysis result, a quantitative score having a value that increases as the degree of abnormality indicated with the diagnosis result indicated with the image diagnosis information 13h increases, as analysis for the image diagnosis information 13h. The score calculation function 14a registers the calculated quantitative score in the item “image diagnosis” in the living body state determination database 13k.

In addition, the score calculation function 14a calculates, as an analysis result, a quantitative score having a value that increases as the degree of abnormality indicated with information relating to the heart indicated with the physiological information 13i increases, as analysis for the physiological information 13i. The score calculation function 14a registers the calculated quantitative score in the item “electrocardiogram” in the living body state determination database 13k.

When the physiological information 13i indicates electroencephalogram information, the score calculation function 14a calculates, as an analysis result, a quantitative score having a value that increases as the degree of abnormality indicated with electroencephalogram information increases. The score calculation function 14a registers the calculated quantitative score in the item “electroencephalograph” (not illustrated) provided in the same manner as the item “electrocardiogram” in the living body state determination database 13k.

In addition, the score calculation function 14a calculates, as an analysis result, a quantitative score having a value that increases as the degree of abnormality indicated with the body findings and symptom indicated with the body findings and symptom information 13j increases, as analysis for the body findings and symptom information 13j. The score calculation function 14a registers the calculated quantitative score in the item “body findings and symptom” in the living body state determination database 13k.

By the method described above, the score calculation function 14a calculates a plurality of quantitative scores corresponding to respective types of living body information, and registers the quantitative scores in the living body state determination database 13k.

The score calculation function 14a does not always automatically calculate the quantitative scores as described above. For example, the score calculation function 14a may receive the quantitative score corresponding to the living body information from the operator via the input interface 11. In addition, the score calculation function 14a may register the received quantitative score in the living body state determination database 13k.

The living body state determination function 14b determines the living body state of the subject, for each of the diseases (disease A to disease N), using the living body state determination database 13k.

When it is described with a specific example, the living body state determination function 14b obtains the correlation coefficient “0.8” indicating correlation between the gene expression and mutation information 13a and the disease A from the living body state determination database 13k illustrated in FIG. 2. The living body state determination function 14b also obtains the quantitative score calculated from the gene expression and mutation information 13a from the item “gene expression and mutation”. The living body state determination function 14b calculates a multiplication value r1 between the obtained quantitative score and the obtained correlation coefficient “0.8”.

Also for each of types of living body information other than the gene expression and mutation information 13a, the living body state determination function 14b obtains the correlation coefficient indicating the type of living body information and the disease A, and the quantitative score calculated from the type of living body information, by the same method. The living body state determination function 14b calculates a multiplication value between the obtained correlation coefficient and the obtained quantitative score in the same manner.

Specifically, the living body state determination function 14b calculates a multiplication value r2 between the quantitative score calculated from the epigenetic environment influence information 13b and the correlation coefficient “0.5”. The living body state determination function 14b also calculates a multiplication value r3 between the quantitative score calculated from the protein expression information 13c and the correlation coefficient “0.65”. The living body state determination function 14b also calculates a multiplication value r4 between the quantitative score calculated from the signal transmission information 13d and the correlation coefficient “0.2”.

The living body state determination function 14b also calculates a multiplication value r5 between the quantitative score calculated from the immune function information 13e and the correlation coefficient “0.4”. The living body state determination function 14b also calculates a multiplication value r6 between the quantitative score calculated from the endocrine function information 13f and the correlation coefficient “0.01”. The living body state determination function 14b also calculates a multiplication value r7 between the quantitative score calculated from the pathological information 13g and the correlation coefficient “0.9”.

The living body state determination function 14b also calculates a multiplication value r8 between the quantitative score calculated from the image diagnosis information 13h and the correlation coefficient “0.3”. The living body state determination function 14b also calculates a multiplication value r9 between the quantitative score calculated from the physiological information 13i and the correlation coefficient “0.0”. The living body state determination function 14b also calculates a multiplication value r10 between the quantitative score calculated from the body findings and symptom information 13j and the correlation coefficient “0.1”.

In addition, the living body state determination function 14b calculates a distance between the origin O of a ten-dimensional coordinate space and the point r, when the point r (r1, r2, r3, r4, r5, r6, r7, r8, r9, r10) is disposed in the ten-dimensional coordinate space. The distance also serves as the size of a vector including (r1, r2, r3, r4, r5, r6, r7, r8, r9, r10) as components.

Thereafter, the living body state determination function 14b normalizes the calculated distance such that the calculated distance has a value falling within the range from “0” to “1”, and calculates the normalized distance as a relation degree. The relation degree is an index indicating the degree with which the subject is related to the disease A, and an index indicating the living body state of the subject with respect to the disease A. More specifically, the relation degree is an index indicating the degree (possibility) with which the subject contracts the disease A, and an index indicating the seriousness of the symptoms of the disease A. As the relation degree becomes close to “1”, the degree with which the subject contracts the disease A increases, and the symptoms of the disease A become more serious. By contrast, as the relation degree becomes close to “0”, the degree with which the subject contracts the disease A decreases, and the symptoms of the disease A become less serious.

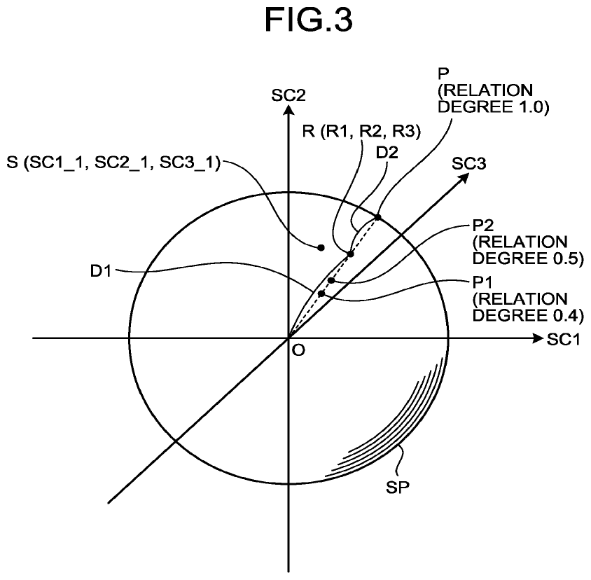

An example of the method for calculating the relation degree from the quantitative scores will now be described with reference to FIG. 3. The case explained above with reference to FIG. 2 is the case where ten quantitative scores generated from ten types of living body information are registered in the living body state determination database 13k, and the living body state determination function 14b calculates the relation degree using the ten quantitative scores. By contrast, in the explanation using FIG. 3, for the sake of convenience of explanation, suppose that three quantitative scores SC1_1, SC2_1, and SC3_1 generated from three types of living body information are registered in the living body state determination database 13k. The explanation using FIG. 3 illustrates an example in which the living body state determination function 14b calculates the relation degree for a single disease using the three quantitative scores SC1_1, SC2_1, and SC3_1.

FIG. 3 is a diagram for explaining an example of the method for calculating the relation degree from the quantitative scores in the first embodiment. FIG. 3 illustrates a three-dimensional space formed of an axis SC1, an axis SC2, and an axis SC3. The axis SC1 is an axis indicating the magnitude of the quantitative score SC1_1. The axis SC2 is an axis indicating the magnitude of the quantitative score SC2_1. The axis SC3 is an axis indicating the magnitude of the quantitative score SC3_1. The three axes SC1, SC2, and SC3 are mutually orthogonal.

In the example of FIG. 3, the living body state determination function 14b calculates a multiplication value R1′ (not illustrated) between the quantitative score SC1_1 and the correlation coefficient corresponding to the quantitative score SC1_1. The living body state determination function 14b also calculates a multiplication value R2′ (not illustrated) between the quantitative score SC2_1 and the correlation coefficient corresponding to the quantitative score SC2_1. The living body state determination function 14b also calculates a multiplication value R3′ (not illustrated) between the quantitative score SC3_1 and the correlation coefficient corresponding to the quantitative score SC3_1.

Thereafter, the living body state determination function 14b calculates a distance between the origin O of the three-dimensional coordinate space and a point R′ when the point R′ (R1′, R2′, R3′) (not illustrated) is disposed in the three-dimensional coordinate space. The distance also serves as the size of a vector including (R1′, R2′, R3′) as components.

Thereafter, the living body state determination function 14b normalizes the calculated distance such that the calculated distance has a value falling within the range from “0” to “1”, and calculates the normalized distance D1 as the relation degree. FIG. 3 illustrates a point R (R1, R2, R3) corresponding to the normalized distance D1. The point R is a point obtained by changing the position of the point R′ with normalization. Specifically, the point R is a point corresponding to the point R′. In FIG. 3, a sphere SP is a sphere having a radius of 1. In the present embodiment, the point R is positioned inside and the surface of the sphere SP.

In addition, as illustrated in FIG. 3, an intersection point of a line segment obtained by further extending the line segment connecting the origin O with the point R from the point R side with the surface of the sphere SP is referred to as point P. In this state, as a distance D2 between the point R and the point P becomes shorter, the degree with which the subject contracts the disease increases, and the symptoms of the disease become more serious.

The explanation with reference to FIG. 3 described above is an explanation of the case of the living body state determination function 14b determining the living body state for the single disease by determining the position of the point R (R1, R2, R3) indicating the current living body state of the subject, in the sphere SP representing the single disease. In the same manner, the living body state determination function 14b also determines the living body state for each of the other diseases. Specifically, the living body state determination function 14b determines the living body state for each of the diseases, by determining the position of the point indicating the current living body state of the subject in each of a plurality of spheres SP corresponding to the respective diseases A to N.

One sphere SP may represent all the diseases A to N. For example, the living body state determination function 14b may determine the living body state for each of all the diseases A to N, by determining the position of the point corresponding to the relation degree of each of the diseases A to N in one sphere SP.

With respect to FIG. 1 again, the living body state determination function 14b determines the living body state of the subject for the disease A on the basis of the calculated relation degree. For example, the living body state determination function 14b determines the living body state by comparing the relation degree with a plurality of thresholds.

For example, when the relation degree falls within a range equal to or larger than the threshold “0” and smaller than “0.4”, it is considered that the subject does not contract the disease A. For this reason, the living body state determination function 14b determines that the living body state of the subject is a state (healthy state) in which the subject does not contract the disease A, when the relation degree falls within the range equal to or larger than the threshold “0” and smaller than “0.4”. The healthy state serves as an example of the first state.

In addition, for example, when the relation degree falls within a range equal to or larger than the threshold “0.6” and smaller than “0.9”, it is considered that the subject contracts the disease A and the symptoms of the disease A of the subject are slight. For this reason, the living body state determination function 14b determines that the living body state of the subject is a state (disease state) in which the subject contracts the disease A and the symptoms of the disease A of the subject are slight, when the relation degree falls within the range equal to or larger than the threshold “0.6” and smaller than “0.9”. The disease state serves as an example of the second state.

In addition, for example, when the relation degree falls within a range equal to or larger than the threshold “0.4” and smaller than “0.6”, it is considered that the living body state of the subject is a state (intermediate state) between the healthy state and the disease state. The intermediate state indicates a state in which the subject changes to the disease state more easily than the “healthy state” described above, although it is a state in which the subject does not contract the disease A. For this reason, the living body state determination function 14b determines that the living body state of the subject is the intermediate state, when the relation degree falls within the range equal to or larger than the threshold “0.4” and smaller than “0.6”. The intermediate state serves as an example of the third state.

In addition, for example, when the relation degree falls within a range equal to or larger than the threshold “0.9” and equal to or smaller than “1.0”, it is considered that the subject contracts the disease A and the symptoms of the disease A of the subject are serious or the subject is dead. For this reason, the living body state determination function 14b determines that the living body state of the subject is a state (serious or dead) in which the subject contracts the disease A and the symptoms of the disease A of the subject are serious or the subject is dead, when the relation degree falls within the range equal to or larger than the threshold “0.9” and equal to or smaller than “1.0”. The serious or dead state serves as an example of the fourth state.

By the method described above, the living body state determination function 14b determines the living body state of the subject with respect to the disease A on the basis of a plurality of quantitative scores and a plurality of correlation coefficients. More specifically, the living body state determination function 14b calculates the relation degree between the subject and the disease A on the basis of a plurality of quantitative scores and a plurality of correlation coefficients, and determines the living body state of the subject with respect to the disease A on the basis of the calculated relation degree. In addition, by the similar method, the living body state determination function 14b calculates the relation degree of the subject with each of the diseases B to N, and determines the living body state of the subject with respect to each of the diseases B to N on the basis of the calculated relation degree. As described above, the living body state determination function 14b determines the living body state of the subject with respect to each of the diseases A to N, on the basis of a plurality of quantitative scores obtained by analysis of a plurality of types of comprehensive living body information ranging from the gene level to the human body level of the subject. Accordingly, the diagnosis support apparatus 10 according to the present embodiment enables determination of the living body state of the subject with high accuracy.

The following is an explanation of the case where the living body state determination function 14b calculates the relation degree for an ischemic heart disease, as the relation degree for the single disease described above, in FIG. 3 explained above. In this case, as illustrated in FIG. 3, the point P on the surface of the sphere SP serves as the point corresponding to the relation degree “1.0” for the ischemic heart disease. In addition, the point P1 serves as the point corresponding to the relation degree “0.4” for the ischemic heart disease. In addition, the point P2 serves as the point corresponding to the relation degree “0.5” for the ischemic heart disease.

For example, there are cases where the subject contracts another disease at the stage before the subject contracts a certain disease. For example, the subject contracts hyperlipemia and arteriosclerotic disease before the subject contracts the ischemic heart disease. Specifically, first, the subject contracts hyperlipemia. When the state in which the subject contracts hyperlipemia continues for certain time, the blood vessels harden, and the subject contracts arteriosclerotic disease. Thereafter, when the state in which the subject contracts the arteriosclerotic disease continues for certain time, the coronary artery is occluded, and the subject contracts the ischemic heart disease.

As described above, the subject in the healthy state does not suddenly contract the ischemic heart disease, but contracts a disease, such as hyperlipemia and arteriosclerotic disease, at the stage before the subject contracts the ischemic heart disease. For this reason, for example, as illustrated in FIG. 3, when the subject contracts hyperlipemia, the relation degree “0.4” corresponding to the point P1 is calculated. In addition, when the subject contracts arteriosclerotic disease, the relation degree “0.5” corresponding to the point P2 is calculated.

For this reason, the living body state determination function 14b may further determine whether the subject contracts a disease other than the ischemic heart disease, on the basis of the relation degree for the ischemic heart disease. Specifically, the living body state determination function 14b may determine the living body state of the subject for a disease other than the ischemic heart disease, on the basis of the relation degree for the ischemic heart disease. For example, when the relation degree for the ischemic heart disease is “0.4”, the living body state determination function 14b may determine that the subject is in the intermediate state for the ischemic heart disease, and in the disease state for hyperlipemia. In addition, when the relation degree for the ischemic heart disease is “0.5”, the living body state determination function 14b may determine that the subject is in the intermediate state for the ischemic heart disease, and in the disease state for the arteriosclerotic disease.

In addition, the living body state determination function 14b calculates total reliability indicating the likelihood of the living body state determined for each of the diseases (disease A to disease N), using the living body state determination database 13k.

First, the following is an explanation of an example of a method for calculating total reliability indicating likelihood of the living body state determined for the disease A. For example, the living body state determination function 14b obtains the correlation coefficient “0.8” corresponding to the gene expression and mutation information 13a for the disease A from the living body state determination database 13k. In addition, the living body state determination function 14b obtains the reliability “A” indicating the likelihood of the obtained correlation coefficient “0.8” from the living body state determination database 13k.

Thereafter, the living body state determination function 14b calculates a multiplication value r11 between the absolute value of the obtained correlation coefficient “0.8” and the obtained reliability “A”.

Also for each of types of living body information other than the gene expression and mutation information 13a, the living body state determination function 14b calculates the correlation coefficient indicating correlation between the type of living body information and the disease A and the reliability indicating the likelihood of the correlation coefficient, by the same method. Thereafter, the living body state determination function 14b calculates a multiplication value between the absolute value of the obtained correlation coefficient and the obtained reliability in the same manner.

Specifically, the living body state determination function 14b calculates a multiplication value r12 between the absolute value of the correlation coefficient “0.5” corresponding to the epigenetic environment influence information 13b and the reliability “B”. The living body state determination function 14b also calculates a multiplication value r13 between the absolute value of the correlation coefficient “0.65” corresponding to the protein expression information 13c and the reliability “A”. The living body state determination function 14b also calculates a multiplication value r14 between the absolute value of the correlation coefficient “0.2” corresponding to the signal transmission information 13d and the reliability “B”.

The living body state determination function 14b also calculates a multiplication value r15 between the absolute value of the correlation coefficient “0.4” corresponding to the immune function information 13e and the reliability “B”. The living body state determination function 14b also calculates a multiplication value r16 between the absolute value of the correlation coefficient “0.01” corresponding to the endocrine function information 13f and the reliability “C”. The living body state determination function 14b also calculates a multiplication value r17 between the absolute value of the correlation coefficient “0.9” corresponding to the pathological information 13g and the reliability “A”.

The living body state determination function 14b also calculates a multiplication value r18 between the absolute value of the correlation coefficient “0.3” corresponding to the image diagnosis information 13h and the reliability “B”. The living body state determination function 14b also calculates a multiplication value r19 between the absolute value of the correlation coefficient “0.0” corresponding to the physiological information 13i and the reliability “C”. The living body state determination function 14b also calculates a multiplication value r20 between the absolute value of the correlation coefficient “0.1” corresponding to the body findings and symptom information 13j and the reliability “B”.

Thereafter, the living body state determination function 14b calculates the sum Q of the ten multiplication values r11 to r20 as the total reliability in accordance with the following expression (1).

Q=r11+r12+r13+r14+r15+r16+r17+r18+r19+r20 (1)

The living body state determination function 14b may use the sum Q as the total reliability without any processing, or may use the value obtained by normalizing the sum Q to any of a plurality of stages A to E, as the total reliability. For example, the total reliability “A” referred to herein is reliability higher than the total reliability “B”, and the total reliability “B” is reliability higher than the total reliability “C”. In addition, the total reliability “C” is reliability higher than the total reliability “D”, and the total reliability “D” is reliability higher than the total reliability “E”. The total reliabilities “A”, “B”, “C”, “D”, and “E” indicate respective numerical values. For example, the total reliability “A” is “1.0”, the total reliability “B” is “0.8”, and the total reliability “C” is “0.6”. In addition, for example, the total reliability “D” is “0.4”, and the total reliability “E” is “0.2”.

By the method described above, the living body state determination function 14b calculates the total reliability indicating likelihood of the living body state determined with respect to the disease A, on the basis of a plurality of reliabilities and a plurality of correlation coefficients. By the same method, the living body state determination function 14b also calculates the total reliability indicating likelihood of the living body state determined with respect to each of the diseases B to N.

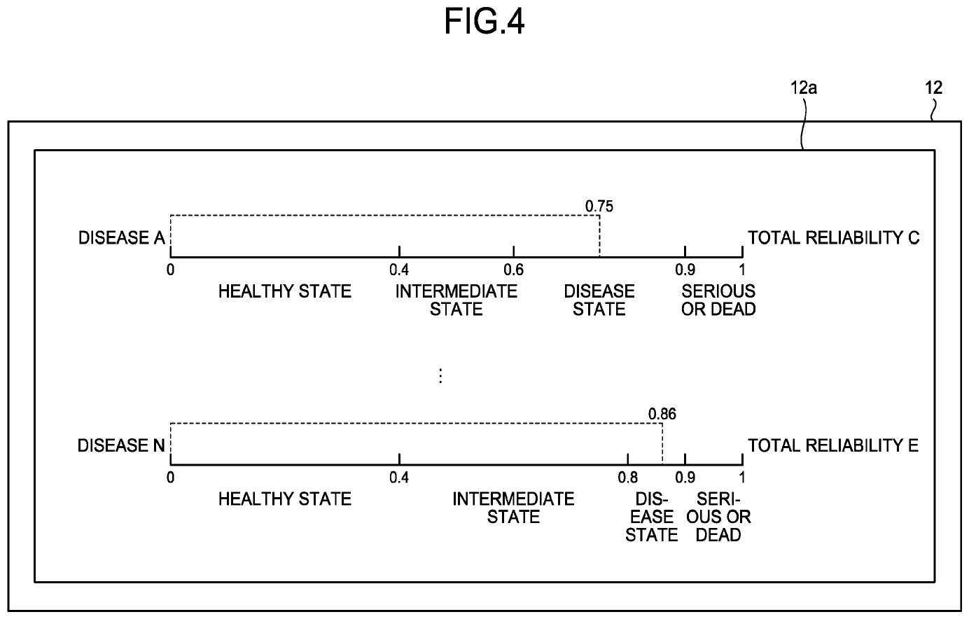

The display control function 14c causes the display 12 to display the relation degrees and the total reliabilities calculated for the respective diseases A to N and the living body states determined for the respective diseases A to N. FIG. 4 is a diagram illustrating an example of display of the relation degrees and the total relation degrees according to the first embodiment.

For example, as illustrated in FIG. 4, the display control function 14c causes the display 12 to display the relation degree “0.75” and the disease state in association with respect to the disease A in a display region 12a. This display enables the operator to easily determine that the living body state of the subject with respect to the disease A is the disease state.

In addition, the display control function 14c causes the display 12 to display the relation degree “0.86” and the disease state in association with respect to the disease N in the display region 12a. This display enables the operator to easily determine that the living body state of the subject with respect to the disease N is the disease state.

In the example of FIG. 4, although illustration of the relation degree and the living body state is omitted for the diseases B to M, the determined living body state and the calculated relation degree are displayed in association in the display region 12a for each of the diseases, in the same manner as the disease A and the disease N.

As described above, the current living body state of the subject is visualized to illustrate to what degree the current living body state is close to each of the diseases A to N. This display enables the operator to provide the subject with health advice and/or support to reduce the relation degree.

In the present embodiment, as described above, the diagnosis support apparatus 10 enables determination of the living body state of the subject with high accuracy. In addition, the diagnosis support apparatus 10 the display 12 to display the accurately determined living body state. With this structure, the diagnosis support apparatus 10 enables the operator to diagnose the disease with high accuracy.

In addition, for example, as illustrated in FIG. 4, the display control function 14c causes the display 12 to display the total reliability “C” for the disease A in the display region 12a. This display enables the operator to recognize that the likelihood of the “disease state” determined for the disease A is “C”.

In addition, the display control function 14c causes the display 12 to display the total reliability “E” for the disease N in the display region 12a. This display enables the operator to recognize that the likelihood of the “disease state” determined for the disease N is “E”.

In the example of FIG. 4, illustration of the total reliabilities for the diseases B to M is omitted, but the total reliabilities thereof are displayed in the display region 12a in the same manner as the disease A to the disease N.

As described above, the diagnosis support apparatus 10 according to the present embodiment causes the displaying of the total reliability indicating likelihood of the determined living body state for each of the diseases. Accordingly, the diagnosis support apparatus 10 according to the present embodiment enables the operator to recognize likelihood of the determined living body state. As a result, for example, the operator is enabled to determine whether execution of any additional inspection is required, in accordance with the total reliability.

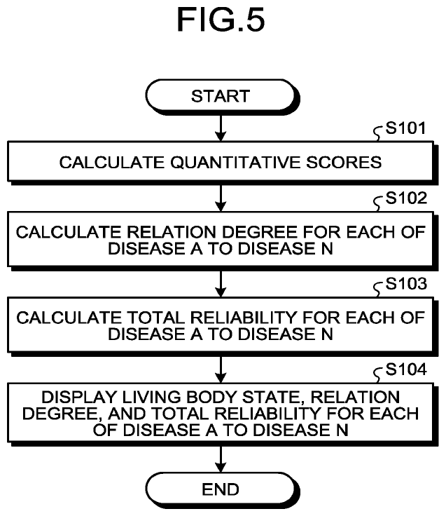

FIG. 5 is a flowchart illustrating a process of an example of processing performed with the diagnosis support apparatus according to the first embodiment. For example, the processing illustrated in FIG. 5 is performed when the operator inputs an instruction to determine the living body state to the processing circuitry 14 via the input interface 11.

As illustrated in FIG. 5, the score calculation function 14a calculates a quantitative score for each of a plurality of types living body information stored in the storage circuit 13 (Step S101). Thereafter, the living body state determination function 14b calculates the relation degree using the quantitative scores and a plurality of correlation coefficients for each of the disease A to the disease N, and determines the living body state on the basis of the relation degree (Step S102).

Thereafter, the living body state determination function 14b calculates the total reliability using the correlation coefficients and the reliabilities for each of the diseases A to N (Step S103). Thereafter, the display control function 14c causes the display 12 to display the living body state, the relation degree, and the total reliability for each of the diseases A to N (Step S104), and ends the processing. For example, at Step S104, the display control function 14c causes the display 12 to display the determined living body state and the relation degree in association as described above.

The diagnosis support apparatus 10 according to the first embodiment has been described above. As described above, the diagnosis support apparatus 10 according to the first embodiment enables determination of the living body state of the subject with high accuracy.

### First Modification of First Embodiment

The following is an explanation of a first modification of the first embodiment. The embodiment described above illustrates the case where the living body state determination function 14b determines which of four living body states of the healthy state, the intermediate state, the disease state, and the serious or dead state the subject is in for each of the disease A to the disease N. However, the method for determining the living body state with the living body state determination function 14b is not limited thereto. For this reason, the first modification illustrates an example of another method for determining the living body state with the living body state determination function 14b.

For example, the living body state determination function 14b according to the first modification may determine which of the healthy state, the intermediate state, and the disease state the subject is in. Specifically, the living body state determination function 14b may have the structure of performing no determination of the serious or dead state for the subject that is clearly in no serious state.

The first modification enables reduction in processing load caused by determination of the living body state, because no determination of the serious or dead state is performed. In addition, the first modification also produces the same effect as that of the first embodiment.

### Second Modification of First Embodiment

In addition, the first embodiment described above has illustrated the case where the living body state determination function 14b determines the living body state using ten correlation coefficients for each of the diseases. However, the living body state determination function 14b may determine the living body state using correlation coefficients each corresponding to reliability equal to or higher than the specific reliability in the ten correlation coefficient. Such a modification will be explained hereinafter as the second modification of the first embodiment. In the explanation of the second modification, points different from the first embodiment will be mainly explained.

The following is an explanation of the case where the reliability “A” is “1.0”, the reliability “B” is “0.7”, the reliability “C” is “0.3”, and the specific reliability is “0.8”. In this case, in the reliabilities “A”, “B”, and “C”, the reliability “A” is reliability equal to or higher than the specific reliability “0.8”. In the second modification, the living body state determination function 14b determines the living body state for each of the diseases A to N using the correlation coefficients corresponding to the reliability “A”.

For example, when the living body state for the disease A is determined, the living body state determination function 14b uses the correlation coefficients each corresponding to the reliability “A” in the living body state determination database 13k illustrated in FIG. 2. For example, the living body state determination function 14b uses the correlation coefficient “0.8” corresponding to the reliability “A”, the correlation coefficient “0.65” corresponding to the reliability “A”, and the correlation coefficient “0.9” corresponding to the reliability “A”, in order from above. Specifically, the living body state determination function 14b calculates the distance between the origin O of the three-dimensional coordinate space and a point r′, when the point r′ (r1, r3, r7) is disposed in the three-dimensional coordinate space. The distance also serves as the magnitude of the vector including (r1, r3, r7) as components.

Thereafter, the living body state determination function 14b normalizes the calculated distance such that the calculated distance has a value falling within the range from “0” to “1”, and calculates the normalized distance as a relation degree, in the same manner as the first embodiment. Thereafter, the living body state determination function 14b determines the living body state for the disease A on the basis of the calculated relation degree, in the same manner as the first embodiment. The living body state determination function 14b also determines the living body state for each of the diseases B to N by the same method.

According to the second modification, the diagnosis support apparatus 10 determines the living body state using the correlation coefficients each corresponding to reliability equal to or higher than the specific reliability. With this structure, the diagnosis support apparatus 10 according to the second modification enables determination of the living body state of the subject with higher accuracy.

### Third Modification of First Embodiment

The first embodiment described above has illustrated the case where the living body state determination function 14b determines the living body state for the disease A by comparing the relation degree with each of the thresholds “0.4”, “0.6”, and “0.9” illustrated in FIG. 4. For example, in FIG. 4, when the relation degree for the disease A is “0.39”, the living body state of the subject is a state of easily changing to the intermediate state, although the subject is determined to be in the healthy state. The same is applicable to the other living body states. Specifically, when the absolute value serving as the difference between the threshold and the relation degree is equal to or smaller than a specific value (for example, 0.2), the subject is in the state of easily changing to a living body state different from the living body state determined with the living body state determination function 14b.

For this reason, in the third modification, the living body state determination function 14b performs the same processing as that of the first embodiment, and performs processing described hereinafter to cause the operator to recognize that the subject is in the state of easily changing to a living body state different from the determined living body state. In the explanation of the third modification, points different from the first embodiment will be mainly explained.

For example, the living body state determination function 14b calculates absolute values of the difference between each of a plurality of thresholds and the relation degree. In this manner, absolute values of a plurality of differences are calculated. Thereafter, the living body state determination function 14b determines whether each of the absolute values of the differences is equal to or smaller than the specific value. When it is determined that at least one of the absolute values of the differences is equal to or smaller than the specific value, the living body state determination function 14b causes the display 12 to display information (warning information) indicating that the subject is in the state of easily changing to a living body state different from the determined living body state. Specifically, the living body state determination function 14b causes the display 12 to display warning information, when the absolute value of the difference between at least one of the threshold and the relation degree is equal to or smaller than the specific value. This structure enables the operator to recognize that the subject is in the state of easily changing to a living body state different from the determined living body state.

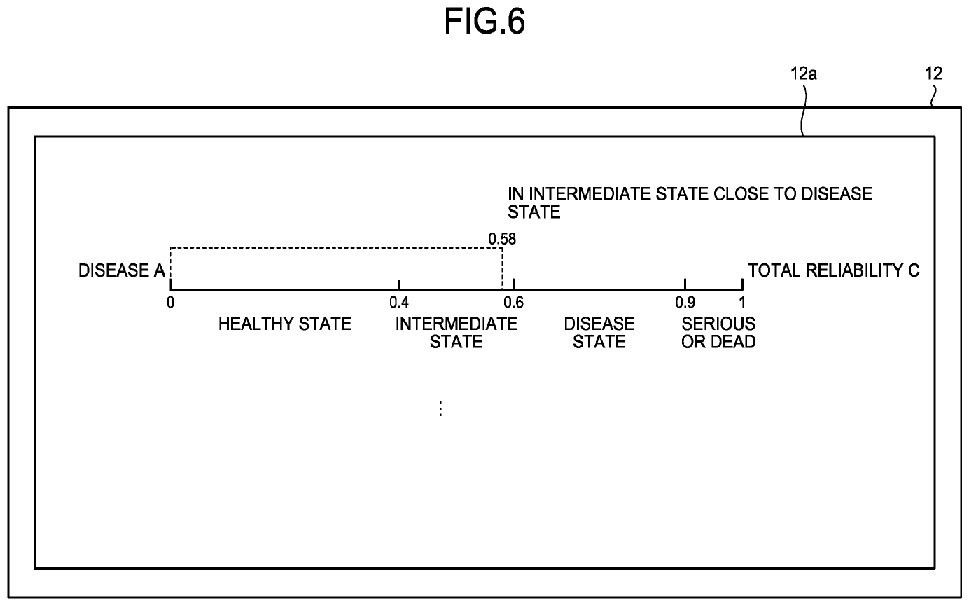

FIG. 6 is a diagram illustrating an example of warning information according to the third modification of the first embodiment. As illustrated in FIG. 6, when the absolute value of the difference between the threshold “0.6” and the relation degree “0.58” is equal to or smaller than the specific value, the living body state determination function 14b generates text data “in the intermediate state close to the disease state” as the warning information, and causes the display 12 to display the generated text data in the display region 12a. This display enables the operator to recognize that the subject is in the state of easily changing to a living body state different from the determined intermediate state.

In particular, in chronic diseases and cancers, it is important to maintain homeostasis to maintain the normal state. Generally, the living body system includes a maintaining mechanism having redundancy and stability to maintain the vital activity. For example, even the states that can be determined by the doctor as the same state to all appearances actually include the state maintained at the healthy state without any problem and the state in which the maintaining mechanism acts to the maximum to maintain the functions somehow.

A complicated system (redundancy mechanism) including a plurality of redundancy paths is used to achieve the living body state and the functions. The redundancy mechanism tends to maintain the state and the functions just before destruction in many cases, while contributing to maintenance of homeostasis, and acts in the direction of advancing deterioration in the disease. For this reason, it is important to determine the detailed state with high accuracy.

In the third modification, for example, even when the living body state of the subject is determined as the disease state, when the absolute value of the difference between the threshold indicating the boundary between the disease state and the serious or dead state and the relation degree is equal to or smaller than the specific value, the operator is enabled to recognize that the subject is in the state of easily changing to the serious or dead state. Specifically, the operator is enabled to recognize information that the maintaining mechanism acts to the maximum to maintain the functions somehow.

### Second Embodiment

A second embodiment will be explained hereinafter. In the second embodiment, the living body state is determined without using quantitative scores. For this reason, in the second embodiment, the processing circuitry 14 includes no score calculation function 14a, but includes the living body state determination function 14b and the display control function 14c.

In the second embodiment, the living body state determination function 14b estimates the living body state using a state equation and an observation equation.

The second embodiment illustrates an example of applying a state spatial model obtained by applying a Karman filter suitable for estimating the quantity hourly changing with lapse of time from observation including discrete errors to the living body being a nonlinear dynamic system to estimation of the current living body state of the subject. For example, in time-series analysis predicting the future from the past data, when the past data does not exist in a time-series manner or includes missing values, prediction is impossible or the prediction accuracy greatly deteriorates. However, there are cases where only discrete data exists, and cases where the observation result does not match the actual state. In the second embodiment, modeling is performed with the state equation and the observation equation separately to construct a flexible model to estimate the observation value and reflect the part having the actual measurement value on the prediction standard. Suppose that the state equation has the Markov property.

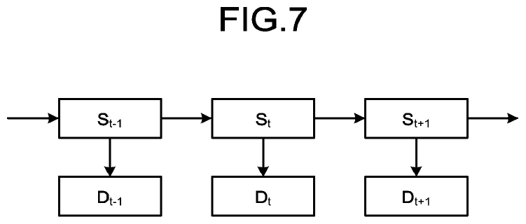

FIG. 7 is a diagram for explaining an example of the method for estimating the living body state according to the second embodiment. FIG. 7 schematically illustrates the state spatial model. In the second embodiment, the living body state determination function 14b estimates the living body state Sₜ of the subject at the current time using the following state equation (2) and the following observation equation (3), as illustrated in FIG. V.

Sₜ=G(Sₜ₋₁,aₜ,uₜ) (2)

Dₜ=F(Gₜ₋₁,bₜ,wₜ) (3)

In the state equation (2) described above, the symbol “Sₜ₋₁” is a variable indicating the living body state of the subject at the time one step before the current time. The symbol “aₜ” is an explanatory variable of the current time. The symbol “uₜ” is a variable indicating system noise of the current time and fluctuation of the living body.

For example, in the present embodiment, each of the four living body states (healthy state, intermediate state, disease state, and serious or dead state) is used in the numerical form. For this reason, “Sₜ₋₁” and “Sₜ” are used as numerical values. The function G is a function to output the living body state Sₜ of the subject at the current time using “Sₜ₋₁”, “aₜ”, and “uₜ”. The initial value S₀ is input to the processing circuitry 14 by the operator via the input interface 11, and stored in the storage circuit 13 with the living body state determination function 14b.

In the observation equation (3) described above, the observation value Dₜ is a variable indicating the living body information of the subject at the current time. For example, the observation value Dₜ is living body information indicating HbAlc obtained by a blood test. The symbol “Gₜ₋₁” is “Sₜ”. The symbol “bₜ” is an explanatory variable at the current time. The symbol “wₜ” is a variable indicating observation noise at the current time. The function F is a function to output the observation value Dₜ using “Gₜ₋₁”, “bₜ”, and “wₜ”.

As illustrated in FIG. 7, incorporating an unobservable state in the model enables construction of a more complicated time-series model of the living body state. In addition, the living body state determination function 14b is enabled to correct the current living body state based on the past living body state on the basis of the current observation value, and determine the current living body state with higher accuracy.

For example, the living body state determination function 14b estimates the corrected living body state S_{A} at the current time with higher accuracy using the following expression (4).

S_{A}=S_{R}+K(D_{R}−D_{P}) (4)

In the expression (4), the symbol “S_{A}” is a variable indicating the corrected living body state at the current time. The symbol “S_{R}” is a variable indicating the living body state at the current time before correction. The symbol “K” is the Kalman gain. The symbol “D_{R}” is an actual observation value. Specifically, “D_{R}” is living body information indicating HbAlc at the current time obtained by an actual blood test. The symbol “D_{P}” is a predicted observation value. Specifically, “D_{F}” is living body information indicating HbAlc at the current time predicted with the observation equation (3).

The living body state determination function 14b is enabled to determine the current living body state with higher accuracy by correcting the current living body state using the expression (4).

In addition, the living body state determination function 14b further corrects the current living body state to match a plurality of quantitative scores, and statistically estimates other unobserved missing values from the state. In this manner, the living body state at the current time is fixed, whether it is observed or not.

When the living body state at the current time is the illness state (disease state or serious or dead state), the operator performs a definite diagnosis, or determines whether to perform any additional inspection or the like when the total reliability is low.

The diagnosis support apparatus 10 according to the second embodiment has been described above. The diagnosis support apparatus 10 according to the second embodiment enables determination of the living body state of the subject with high accuracy using the state equation and the observation equation.

### Third Embodiment

The first embodiment has explained the case where the living body state determination function 14b calculates the relation degree using ten quantitative scores and ten correlation coefficients for each of the diseases. However, the living body state determination function 14b may derive the relation degree from ten quantitative scores using a learned model, instead of ten correlation coefficients. Such an embodiment will be explained as a third embodiment hereinafter.

In the third embodiment, learned models corresponding to the respective diseases A to N are stored in the storage circuit 13. Specifically, fourteen learned models are stored in the storage circuit 13. Each of the learned models outputs the living body state in response to input of ten quantitative scores.

For example, the learned model corresponding to the disease A may be generated with the diagnosis support apparatus 10, or generated with a device other than the diagnosis support apparatus 10. The following is an explanation of the case where a device other than the diagnosis support apparatus 10 generates the learned model corresponding to the disease A. In the following explanation, the device is expressed as learned model generation device.

The learned model generation device generates a learned model corresponding to the disease A by learning relation between a combination of a plurality of pieces of living body information of the subject and the living body state for the disease A. The pieces of living body information herein are the gene expression and mutation information 13a, the epigenetic environment influence information 13b, the protein expression information 13c, the signal transmission information 13d, the immune function information 13e, the endocrine function information 13f, the pathological information 13g, the image diagnosis information 13h, the physiological information 13i, and the body findings and symptom information 13j.

For example, the learned model generation device performs machine learning by inputting a pair of the combination and the living body state as learning data (teacher data) to the machine learning engine.

As a result of machine learning as described above, the learned model generation device generates a learned model to output the living body state in response to input of the combination. As described above, the learned model generation device generates a learned model using a plurality of types of comprehensive living body information ranging from the gene level to the human body level of the subject. With this structure, the learned model generation device is enabled to generate a learned model to output the living body state with high accuracy. Thereafter, the learned model generation device transmits the generated learned model to the diagnosis support apparatus 10 via a network that is not illustrated. The diagnosis support apparatus 10 stores the received learned model in the storage circuit 13.

Thereafter, the living body state determination function 14b of the diagnosis support apparatus 10 derives living body information by inputting ten quantitative scores to the learned model corresponding to the disease A. By the same method, the living body state determination function 14b derives living body information for each of the diseases B to N by inputting ten quantitative scores to the learned model corresponding to each of the diseases B to N.

The diagnosis support apparatus 10 according to the third embodiment has been described above. The diagnosis support apparatus 10 according to the third embodiment enables determination of the living body state of the subject with high accuracy using the learned model.

At least one of the embodiments and the modifications described above enables determination of the living body state of the subject with high accuracy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A diagnosis support apparatus comprising:
a memory configured to store therein a plurality of types of living body information including gene expression and mutation information, epigenetic environment influence information, protein expression information, signal transmission information, immune function information, endocrine function information, pathological information, image diagnosis information, physiological information, and body findings and symptom information of a subject; and
processing circuitry configured to determine a living body state of the subject on the basis of a plurality of analysis results obtained by analysis of the types of living body information.

2. The diagnosis support apparatus according to claim 1, wherein the processing circuitry obtains the analysis results by performing the analysis on the types of living body information, and determines the living body state on the basis of the analysis results.

3. The diagnosis support apparatus according to claim 2, wherein the processing circuitry determines the living body state on the basis of the analysis results and correlation coefficients indicating correlation between a disease and the respective types of living body information.

4. The diagnosis support apparatus according to claim 3, wherein the processing circuitry calculates a relation degree between the subject and the disease on the basis of the analysis results and the correlation coefficients, and determines the living body state on the basis of the calculated relation degree.

5. The diagnosis support apparatus according to claim 3, wherein the processing circuitry determines which of at least a first state, a second state, and a third state the subject is in, as the living body state, the first state being a state in which the subject does not contract the disease, the second state being a state in which the subject contracts the disease, and the third state being a state between the first state and the second state.

6. The diagnosis support apparatus according to claim 3, wherein the processing circuitry determines the living body state for each of a plurality of the diseases.

7. The diagnosis support apparatus according to claim 3, wherein the processing circuitry calculates total reliability indicating likelihood of the living body state on the basis of reliabilities of the respective correlation coefficients and the correlation coefficients.

8. The diagnosis support apparatus according to claim 6, wherein the processing circuitry calculates, for each of the diseases, total reliability indicating likelihood of the living body state on the basis of reliabilities of the respective correlation coefficients and the correlation coefficients.

9. The diagnosis support apparatus according to claim 7, wherein the processing circuitry determines the living body state using, in the correlation coefficients, correlation coefficients each corresponding to reliability higher than a specific reliability.

10. The diagnosis support apparatus according to claim 4, wherein
the processing circuitry determines the living body state by comparing a plurality of thresholds with the relation degree, and
calculates information indicating that the subject is in a state of easily changing to a living body state different from the determined living body state, when an absolute value of a difference between any one of the thresholds and the relation degree is equal to or smaller than a specific value.

11. The diagnosis support apparatus according to claim 4, wherein the processing circuitry determines the living body state of the subject for diseases other than the disease on the basis of the relation degree.

12. The diagnosis support apparatus according to claim 1, wherein the processing circuitry causes a display to display the living body state.

13. The diagnosis support apparatus according to claim 4, wherein the processing circuitry causes a display to display the relation degree.

14. The diagnosis support apparatus according to claim 7, wherein the processing circuitry causes a display to display the living body state and the total reliability.

15. The diagnosis support apparatus according to claim 10, wherein the processing circuitry causes a display to display the information.

16. The diagnosis support apparatus according to claim 1, wherein the memory stores therein the types of living body information including living body information of a gene, living body information relating to protein, living body information relating to signal transmission, living body information relating to an endocrine function, living body information relating to an immune function, living body information relating to environment influence, and living body information relating to human body of the subject.
